(19) United States
(12) Patent Application Publication
(10) Pub. No.: US 2017/0266154 A1
(43) Pub. Date: Sep. 21, 2017

(54) METHOD FOR TREATING ALZHEIMER'S DISEASE AND 2, 3-BIPHOSPHOGLYCERATE METABOLISM DISORDER INDUCED

(71) Applicant: National Sun Yat-sen University, Kaohsiung (TW)

(72) Inventors: Chia-Chen Wang, Kaohsiung (TW); Wei-Ren Chen, Kaohsiung (TW)

(73) Assignee: National Sun Yat-sen University, Kaohsiung (TW)

(21) Appl. No.: 15/071,179

(22) Filed: Mar. 15, 2016

(51) Int. Cl. A61K 31/365; A61K 31/6615

(52) U.S. Cl. CPC A61K 31/365; A61K 31/6615

(57) ABSTRACT

The present invention relates to a method for treating Alzheimer's disease and 2,3-BPG metabolic disorder induced morbidities, comprising administrating a phthalide compound to an Alzheimer's disease patient or a patient having 2,3-BPG metabolic disorder induced morbidities, wherein the method is characterized by that the phthalide compound has the same effect as 2,3-BPG on modulating hemoglobin to reduce its oxygen affinity and can thus act as a 2,3-BPG functional substitute when the 2,3-BPG concentration is too low in the Alzheimer's disease patient or in the patient having 2,3-BPG metabolic disorder induced morbidities to maintain the normal oxygen release function of hemoglobin and therefore to maintain the normal cellular oxygenation level.

### FIELD OF THE INVENTION

The present invention is in the medical field, relating to a method for treating Alzheimer's disease and 2,3-biphosphoglycerate metabolic disorders.

### BACKGROUND OF THE INVENTION

Alzheimer's disease (AD) is a common neurodegenerative disease, which accounts for 60% to 70% of dementia cases. Nearly forty million people are affected by Alzheimer's disease nowadays, and it is estimated that by 2050, more than one hundred million people worldwide will suffer from Alzheimer's disease. Yet there remains no effective medications for treating Alzheimer's disease Alzheimer's disease may be caused by metabolic disorders in the brain due to aging. These metabolic disorders can be used as biomarkers for diagnosing Alzheimer's disease. Increasingly more scientific evidence has confirmed that, in addition to the brain and its peripheral tissues, other cells such as erythrocytes, thrombocytes and leukocytes of Alzheimer's disease patients are also impaired. The erythrocytes of Alzheimer's disease patients exhibit altered cellular morphology and some functional defects, changing the ability of the erythrocytes to pass through microcirculation. Recent studies have also found that the blood oxygenation level of the Alzheimer's disease patients in the parietal lobe, which controls movement, intuition and perception, and the frontal lobe, which controls thinking, conception, emotion and integration are significantly low, damaging regions of the brain which control the corresponding functions and leading to malfunctions in these brain regions.

Hemoglobin (Hb), the oxygen-carrying protein in erythrocytes transports oxygen from respiratory organs such as respiratory tracts and lungs and releases oxygen to organs and peripheral tissues of a human body such that the organs and the peripheral tissues can be supplied with sufficient oxygen in order to maintain their normal physiological functions.

Hemoglobin of human adults is a tetramer α₂β₂ consisting of four subunits, α₁, α₂, β₁ and β₂, wherein each subunit relies on intermolecular interactions such as intra-subunit hydrogen bonds to sustain its secondary and tertiary structures. Additionally, the inter-subunit hydrogen bonds formed among the aforementioned four subunits allow hemoglobin to form a quaternary structure.

Hemoglobin can reside in two different quaternary configurations, including the relaxed form (R form) having high oxygen affinity and the tense form (T form) having low oxygen affinity. When hemoglobin is travelled to lungs through the blood circulation, hemoglobin becomes bound with oxygen and resides in the R quaternary configuration of high oxygen affinity. The oxygenated hemoglobin is then transported to organs and peripheral tissues through blood circulation and releases oxygen to organs and peripheral tissues and transforms into the T quaternary configuration of low oxygen affinity. The allostery of hemoglobin is also influenced by several allosteric factors, such as the pH value, the carbon dioxide concentration and the 2,3-BPG concentration in erythrocytes.

2,3-bisphosphorglycerate (2,3-BPG, or 2,3-diphosphoglycerate (2,3-DPG), hereinafter “2,3-BPG”) is the endogenous allosteric effector of hemoglobin and the most important chemical species in an erythrocyte of a human body besides the oxygen-carrying entity, hemoglobin. 2,3-BPG delicately regulates the configuration of hemoglobin by interacting with the β₁ and β₂ subunits of hemoglobin to stabilize hemoglobin in the low oxygen affinity T form to reduce the oxygen affinity of hemoglobin, thereby facilitating hemoglobin to effectively release oxygen to body organs and tissue cells.

When the 2,3-BPG metabolic disorders occur and the 2,3-BPG concentration becomes too low, hemoglobin is unable to release oxygen properly, and the low oxygenation level of organs and tissue cells will likely cause diseases including Alzheimer's disease or other 2,3-BPG metabolic disorder induced morbidities.

Y. G. Kaminsky et al. (Aging Dis, 2013, 4 (5):244-255.) disclose that the 2,3-BPG level of Alzheimer's disease patients decreases significantly. In addition, Elena A. Kosenko et al. (CNS & Neurological Disorders—Drug Targets, 2016, 15, 113-123) also indicate that the Alzheimer's disease patients exhibit significantly low 2,3-BPG concentration and 2,3-BPG metabolic disorders. Accordingly, the goal of the present invention is to improve hypoxia symptoms caused by 2,3-BPG metabolic disorders and thus to treat 2,3-BPG disorder induced Alzheimer's disease.

### BRIEF DESCRIPTION OF THE DRAWINGS

FIG. 1 shows the 2,3-BPG level of subjects in each group; YC: Young adult control group; AC: Age-matched control group matching the Alzheimer's disease patient group; AD: Alzheimer's disease patient group; NA: non-AD dementia patient group.

FIG. 2 shows the synergistic effect of phthalide compounds and 2,3-BPG; A: Z-Ligustilide; B: Senkyunolide I.

FIG. 3 shows oxygen equilibrium curves for hemoglobin under various levels of 2,3-BPG (0.2-12 mM), illustrating that when the 2,3-BPG concentration is higher, the oxygen equilibrium curve for hemoglobin moves more toward the right, and the P₅₀ value is higher. The curves from the left to the right respectively represent: pure hemoglobin (Pure Hb) (control group), 0.6 mM 2,3-BPG, 1.2 mM 2,3-BPG, 4.0 mM 2,3-BPG, 8.0 mM 2,3-BPG and 12.0 mM 2,3-BPG.

FIG. 4 shows oxygen equilibrium curves for hemoglobin under various 2,3-BPG concentrations to illustrate changes in the oxygen saturation fraction of human brain tissues, ordinary cells and alveoli at various physiological oxygen partial pressures when being modulated by 2,3-BPG. The curves from the left to the right respectively represent: pure hemoglobin (Pure Hb) (control group), 0.6 mM 2,3-BPG, 1.2 mM 2,3-BPG, 4.0 mM 2,3-BPG, 8.0 mM 2,3-BPG and 12.0 mM 2,3-BPG.

FIG. 5 shows the effect of the concentrations (mM) of various phthalide compounds on the P₅₀ value of hemoglobin, illustrating that the P₅₀ value of hemoglobin is increased with increasing concentrations of phthalide compounds, which means the oxygen affinity of hemoglobin is reduced and the oxygen release efficiency of hemoglobin is increased.

FIG. 6 shows the effect of the concentrations (mM) of various phthalide compounds on the P₅₀ value of hemoglobin under the presence of various concentrations of 2,3-BPG, illustrating that various phthalide compounds can aid hemoglobin to achieve its physiological P₅₀ value even at low 2,3-BPG levels.

FIG. 7 shows the phthalide compound can enhance the effect of 2,3-BPG to decrease the oxygen saturation fraction of hemoglobin and increase the oxygen release efficiency. The curves from the left to the right respectively represent: pure hemoglobin (Pure Hb) (control group), 1.2 mM 2,3-BPG, 1.2 mM 2,3-BPG and 1.2 mM phthalide compound, and 1.2 mM 2,3-BPG and 4.0 mM phthalide compound.

FIGS. 8A-8L show the chemical structure of twelve phthalide compounds; 8A: Z-butylidenephthalide; 8B: Z-ligustilide; 8C: senkyunolide A; 8D: senkyunolide H; 8E: senkyunolide I; 8F: senkyunolide F; 8G: E-butylidenephthalide; 8H: E-ligustilide; 8I: 3-butylphthalide; 8J: 3-butylidene-4-hydrophthalide; 8K: 6,7-dihydroxyligustilide.

FIG. 9 is a diagram, showing the molecular structure of the functional groups of phthalide compounds.

### SUMMARY OF THE INVENTION

The present invention relates to a method for treating Alzheimer's disease and 2,3-BPG metabolic disorder induced morbidities, comprising administering a phthalide compound to an Alzheimer's disease patient or a patient having 2,3-BPG metabolic disorder induced morbidities, wherein the method is characterized by that the phthalide compound has the same effect as 2,3-BPG on modulating hemoglobin to reduce its oxygen affinity and acts as a substitute for 2,3-BPG when the 2,3-BPG concentration is too low for the Alzheimer's disease patient or the patient having 2,3-BPG metabolic disorder induced morbidities to maintain the biological function of hemoglobin for normal release of oxygen to tissue cells and to maintain the cellular oxygenation level within a normal range.

### DETAILED DESCRIPTION OF THE INVENTION

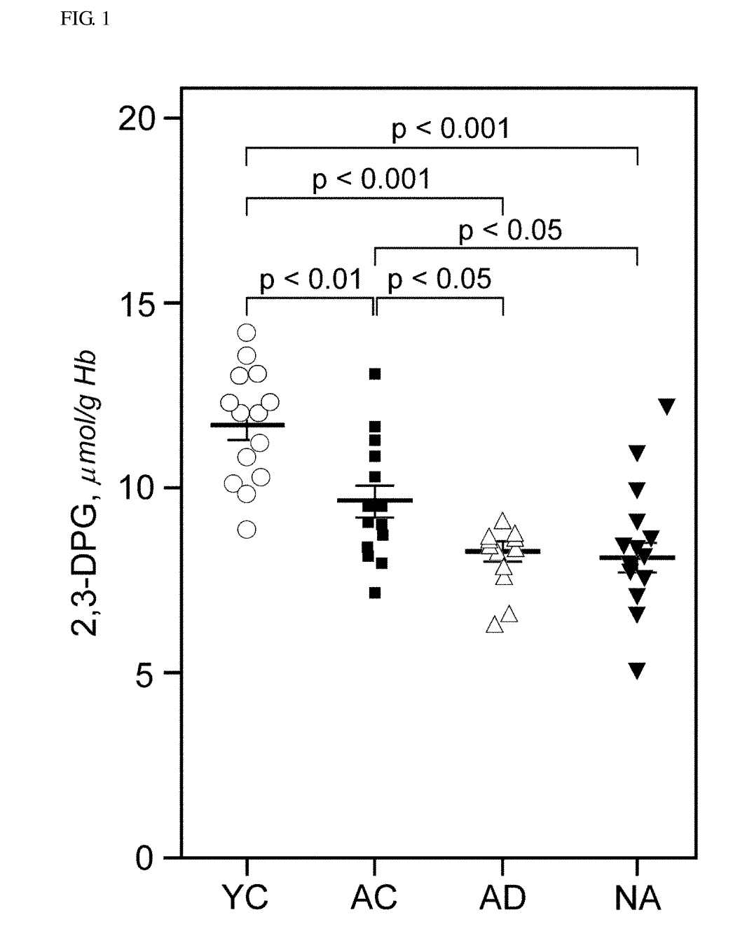

The metabolism of 2,3-BPG can be disturbed with chronic aging, resulting in low level of 2,3-BPG, high oxygen affinity of hemoglobin, low oxygen release efficiency to cells and tissues, low cellular oxygenation level and dysfunction of organs, which eventually lead to an increased incidence rate of various morbidities, for example Alzheimer's disease. Y.bG. Kaminsky et al. (Aging Dis 2013 October; 4 (5): 244-255) disclose that the 2,3-BPG concentration in Alzheimer's disease (AD) patients decreases significantly when compared to younger people and people of the same age (FIG. 1). In addition, Elena A. Kosenko et al. (CNS & Neurological Disorders—Drug Targets, 2016, 15, 113-123) also disclose that Alzheimer's disease (AD) patients have significantly low 2,3-BPG concentration and metabolic disorders. Arai, H. et al. and Hock, C. et al examine the frontal lobe (controlling functions such as thinking, conception, emotion and integration) and the parietal lobe (controlling functions such as movement, intuition and perception) of Alzheimer's disease (AD) patients, concluding that the cerebral hemoglobin oxygenation level in these brain regions decreases significantly, resulting in abnormal brain function. Accordingly, the goal of the present invention is to use a phthalide compound as a functional substitute for 2,3-BPG, to remedy the inefficient oxygen transport of hemoglobin (Hb) caused by low 2,3-BPG concentration, and eventually to treat Alzheimer's disease, 2,3-BPG metabolic disorder induced morbidities, or other non-AD dementia.

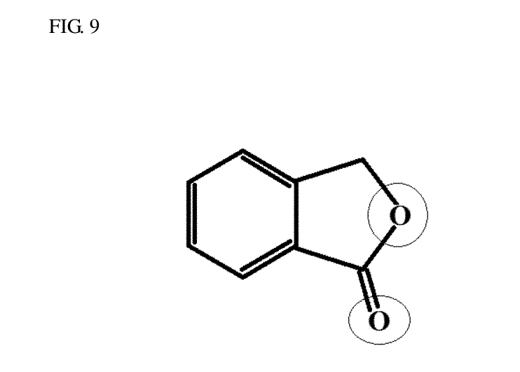

The present invention also provides a method for treating 2,3-BPG metabolic disorders by using a phthalide compound, comprising of administering the phthalide compound to a patient having 2,3-BPG metabolic disorder, wherein the method is characterized by that the phthalide compound has a similar effect as 2,3-BPG on modulating hemoglobin to reduce its oxygen affinity and acts as a functional substitute for 2,3-BPG when the 2,3-BPG concentration is too low for the patient to maintain the biological function of hemoglobin in releasing oxygen to tissue cells and to maintain the cellular oxygenation level in a normal range in order to alleviate the consequences caused by such 2,3-BPG disorder. The phthalide compound can be any compound which exhibits the structural characteristics of the functional groups of the phthalide compounds as shown in FIG. 9, wherein the circled areas are the functional groups of the phthalide compound, which is characterized by an endocyclic oxygen atom and an adjacent ketone group.

The present invention provides a method for treating Alzheimer's disease by using a phthalide compound, comprising administering the phthalide compound to a Alzheimer's disease patient, wherein the method is characterized by that the phthalide compound exhibits a similar effect as 2,3-BPG on modulating hemoglobin to reduce its oxygen affinity and can act as a substitute for 2,3-BPG when the 2,3-BPG concentration is too low in the Alzheimer's disease patient to fulfill the biological function of hemoglobin in releasing oxygen to tissue cells by supplying the phthalide compound and to maintain cellular oxygenation level in a normal range for the treatment of the Alzheimer's disease. The phthalide compound can be any compound which exhibits the structural features of the functional groups of the phthalide compounds as shown in FIG. 9, wherein the circled areas are the functional groups of the phthalide compound, which is characterized by an endocyclic oxygen atom and an adjacent ketone group.

In addition to the phthalide compound, the method provided by the present invention can also adjunctly administer 2,3-BPG to an Alzheimer's disease patient or a patient having 2,3-BPG metabolic disorder induced morbidities.

The 2,3-BPG concentration in healthy people at the sea level is about 5 mM. When the 2,3-BPG metabolism is perturbed, the 2,3-BPG concentration in a human body decreases, and hemoglobin becomes unable to release oxygen to organs and tissue cells easily. Therefore, the present invention further provides a method for preparing a phthalide compound for treating 2,3-BPG metabolic disorder induced morbidities, wherein the phthalide compound is used as a substitute for 2,3-BPG or to compensate for the low 2,3-BPG concentration in patients having 2,3-BPG metabolic disorders, to facilitate the biological function of hemoglobin in releasing oxygen in order to treat or ameliorate the morbidities.

In one preferred embodiment, the phthalide compound has the ability to inhibit the transformation of oxygenated hemoglobin into the R form, thereby stabilizing the oxygen-carrying hemoglobin in the T form which has low oxygen affinity and can release oxygen readily.

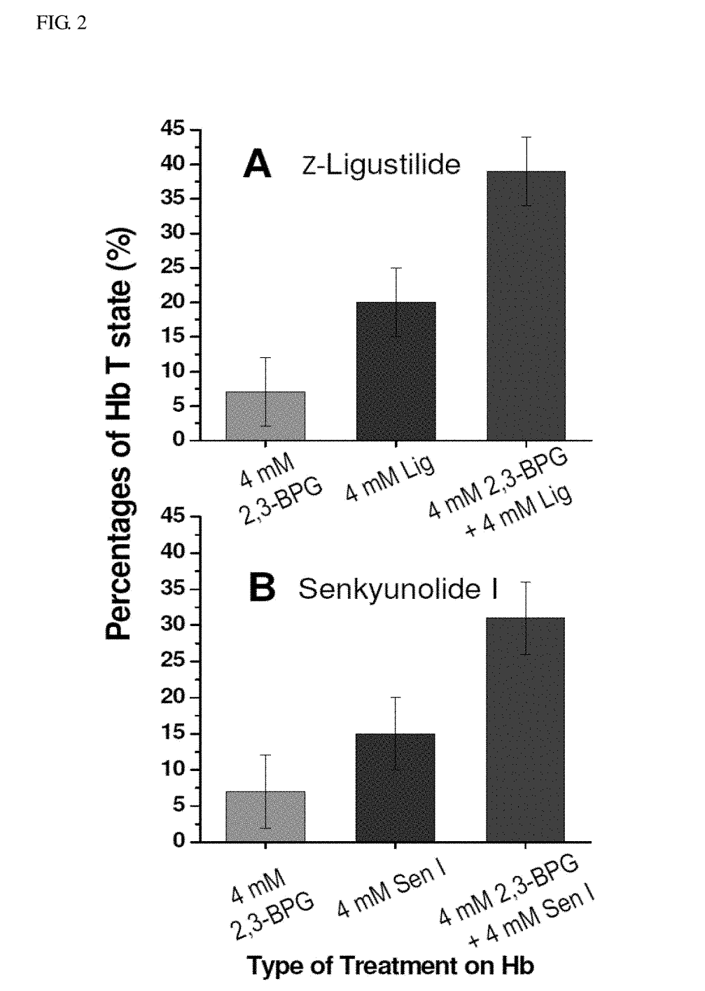

The phthalide compound provided by the present invention is not only used to substitute for 2,3-BPG or to compensate for the low 2,3-BPG concentration in Alzheimer's disease patients and patients having 2,3-BPG metabolic disorder induced morbidities, but also to cooperate with 2,3-BPG to provide a synergistic hemoglobin modulating effect (as shown in FIG. 2).

Therefore, the method of the present invention is to use a drug, which is prepared by using the phthalide compound to cooperate or to act as a substitute for 2,3-BPG to increase the oxygen release efficiency of hemoglobin (Hb) and to improve oxygen transport efficiency when the 2,3-BPG concentration becomes deficient due to 2,3-BPG metabolic disorders, for the treatment of Alzheimer's disease and 2,3-BPG metabolic disorder induced morbidities. The method of the present invention provides a synergistic effect with the 2,3-BPG, either inside the patient body or additionally administered via other 2,3-BPG sources.

### EXAMPLES

The present invention may be embodied it different forms and is not limited by the examples mentioned in the following text. Those of ordinary skill in the art will recognize that many obvious modifications may be made thereto without departing from the sprit or scope of the present invention,

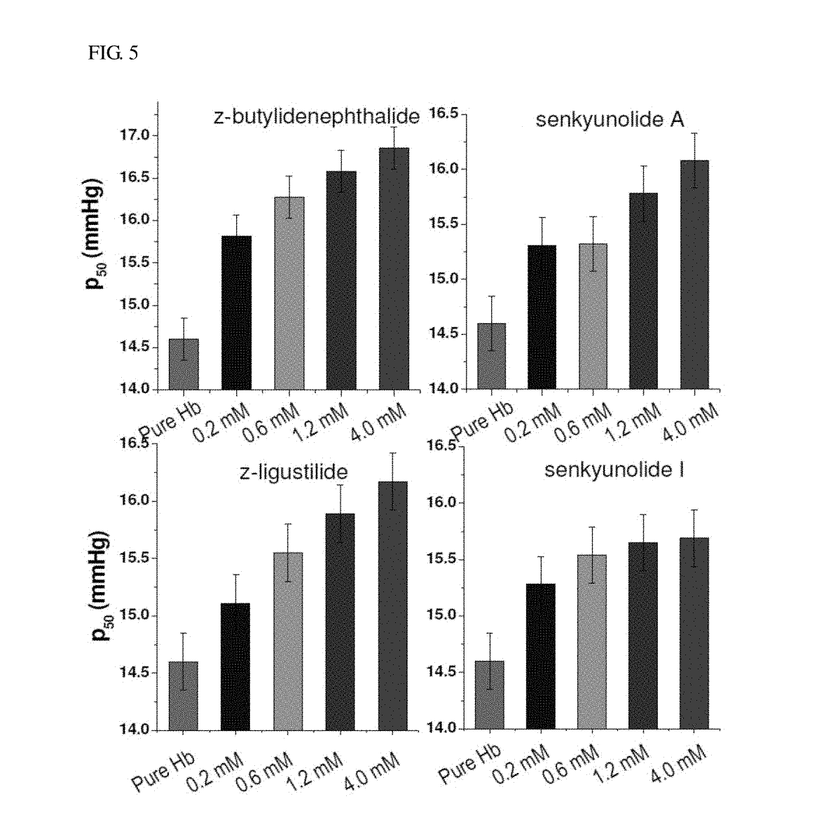

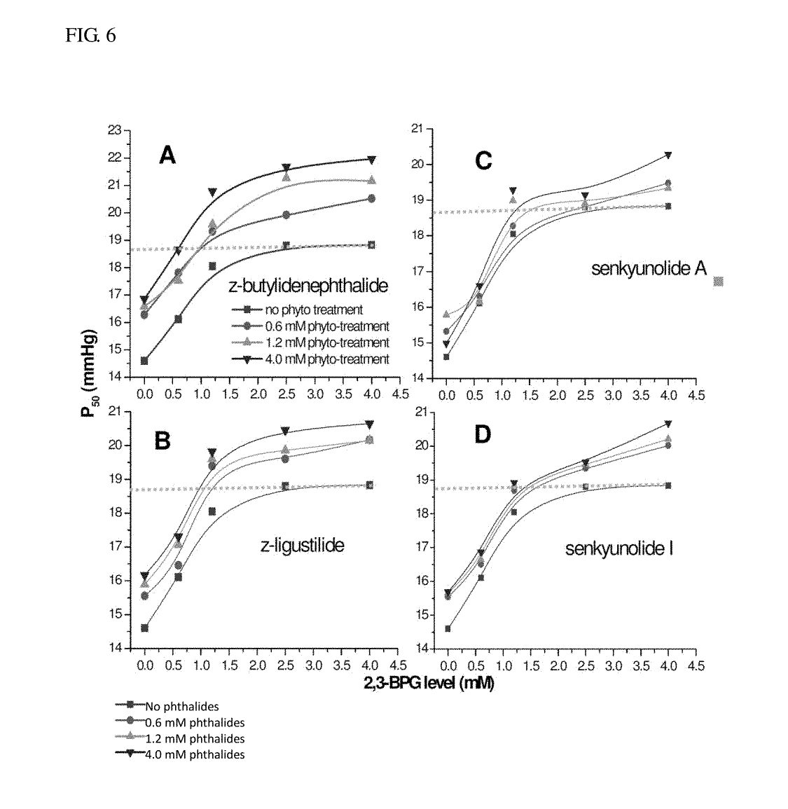

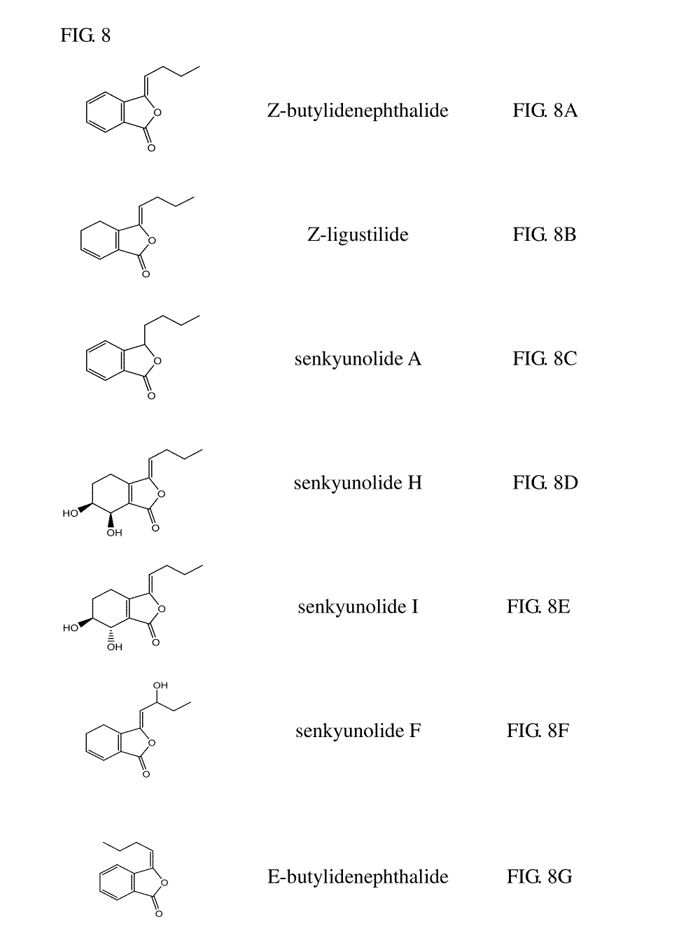

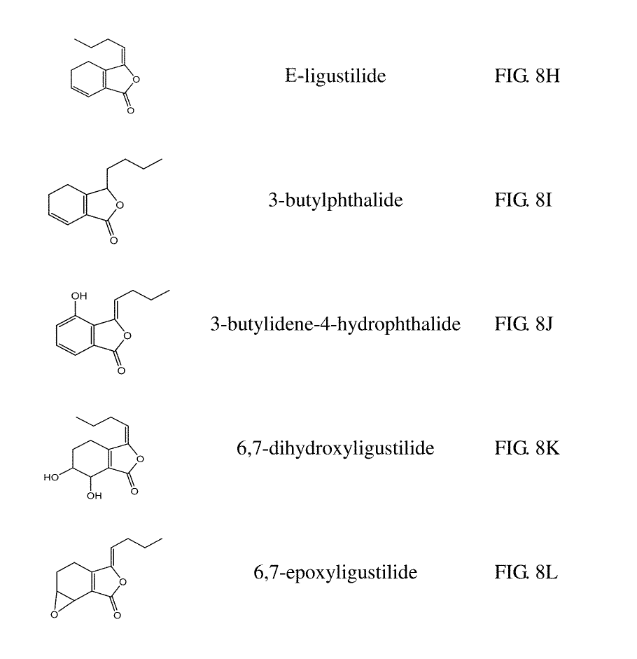

The phthalide compounds provided by the present invention could be any compounds comprising the functional structural features of the phthalide compound, for example, Z-butylidenephthalide (as shown in FIG. 8A), Z-ligustilide (as shown in FIG. 8B), senkyunolide A (as shown in FIG. 8C), senkyunolide H (as shown in FIG. 8D), senkyunolide I (as shown in FIG. 8E), senkyunolide F (as shown in FIG. 8F), E-butylidenephthalide (as shown in FIG. 8G), E-ligustilide (as shown in FIG. 8H), 3-butylphthalide (as shown in FIG. 8I), 3-butylidene-4-hydrophthalide (as shown in FIG. 8J), 6,7-dihydroxyligustilide (as shown in FIG. 8K) and 6,7-epoxyligustilide (as shown in FIG. 8L).

The phthalide compounds could be used in combination with other compounds which could stabilize the oxygen-bound hemoglobin in the T form, for example, 2,3-BPG.

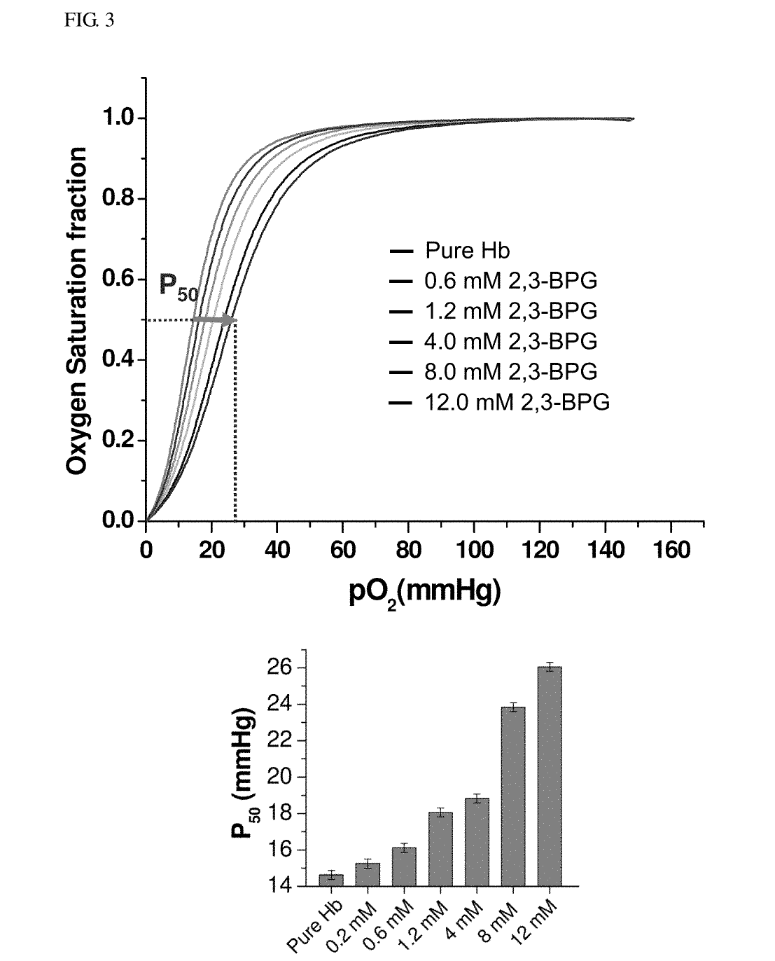

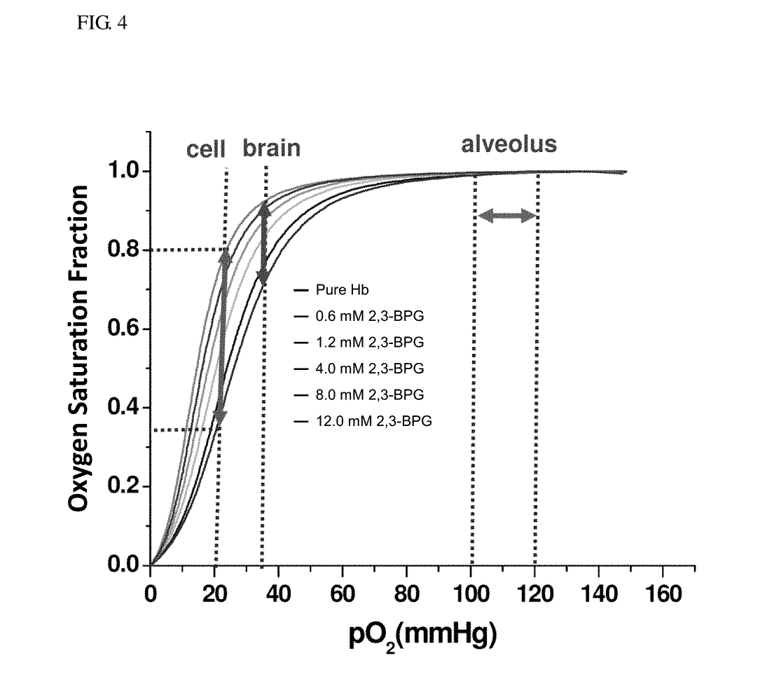

The oxygen affinity of hemoglobin was represented by P₅₀ value. The P₅₀ value was the required partial pressure of oxygen to achieve 50% oxygen saturation. The P₅₀ value of a normal adult was approximately 3.59 kPa (27 mmHg). An increased blood P_{CO2}, a decreased pH value or an increased 2,3-BPG concentration in erythrocytes could reduce the oxygen affinity of hemoglobin, shift the oxygen equilibrium curve to the right and increase the P₅₀ value (as shown in FIG. 3). On the contrary, when the oxygen affinity of hemoglobin was increased, the oxygen equilibrium curve was shifted to the left, and the P₅₀ value was decreased. Under normal conditions, the P_{O2} (oxygen partial pressure) of human cells was approximately 9.9-19 mmHg (J. Cell. Mol. Med., 15, 1239-1253 (2011)). By observing the oxygen saturation fraction of hemoglobin treated with varying concentrations of 2,3-BPG at a fixed oxygen partial pressure through hemoglobin-oxygen equilibrium curves (as shown in FIG. 4), the effect of 2,3-BPG on increasing the oxygen release efficiency of hemoglobin could be more clearly understood. At the oxygen partial pressure of 20 mmHg, the oxygen saturation fraction of the hemoglobin treated with 12 mM of 2,3-BPG to hemoglobin decreased from about 80% to 35%, as compared with hemoglobin without any 2,3-BPG treatment, i.e., the oxygen release efficiency of hemoglobin was increased from 20% to 65%.

In one preferred example, the phthalide compound could effectively decrease the oxygen affinity of the hemoglobin. The P₅₀ value of hemoglobin was increased with increasing concentration of the phthalide compounds, while the oxygen affinity of hemoglobin was lowered (as shown in FIG. 5).

In another example, when there was no phthalide compound, about 4 mM of 2,3-BPG was required for hemoglobin to reach a P₅₀ value of 18.8 mmHg; but after the phthalide compounds were administered, only about 0.6-1.2 mM of 2,3-BPG was required to reach approximately the same or higher P₅₀ value (as shown in FIG. 6).

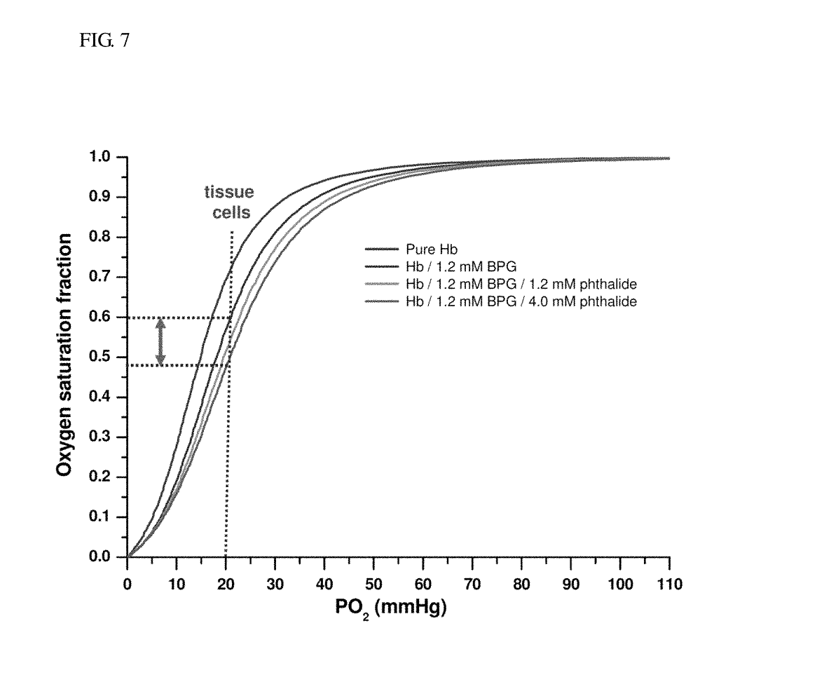

In another example, as shown in FIG. 7, when PO₂ was 20 mmHg and the 2,3-BPG concentration was 1.2 mM, the oxygen saturation level was about 60%. After treating with a phthalide compound, however, the oxygen saturation fraction decreased to about 47%, i.e., the oxygen release efficiency was increased from 40% to 53%. Therefore, it was confirmed that the phthalide compound was able to synergistically enhance the effect of 2,3-BPG, including that already existed in erythrocytes of a patient or that additionally administered, thus promoting the oxygen release efficiency from hemoglobin.

In another example, a drug prepared by using the phthalide compound could be administered adjunctly with 2,3-BPG to a Alzheimer's disease patient, wherein the administering method includes oral administration, injection and inhalation of aerosolized medication

In summary, the present invention provided a method of using a phthalide compound which had the same effect as 2,3-BPG on modulating hemoglobin to reduce its oxygen affinity and acted as a 2,3-BPG functional substitute to compensate for the insufficient 2,3-BPG to maintain the biological function of hemoglobin for normally releasing oxygen to tissues and cells, when the 2,3-BPG concentration was too low in Alzheimer's disease patients or in patients having 2,3-BPG metabolic disorder induced morbidities, for maintaining the cellular oxygenation level in a normal range and for treating or preventing Alzheimer's disease and 2,3-BPG metabolic disorder induced morbidities.

What is claimed is:

1. A method for treating Alzheimer's disease, comprising administrating a phthalide compound to an Alzheimer's disease patient, wherein the phthalide compound substitutes for or cooperate synergistically with 2,3-BPG in the Alzheimer's disease patient to increase the oxygen release efficiency of hemoglobin in the brain of the patient.

2. The method of claim 1, wherein the phthalide compound is selected from the group consisting of Z-butylidenephthalide, Z-ligustilide, senkyunolide A, senkyunolide H, senkyunolide I, senkyunolide F, E-butylidenephthalide, E-ligustilide, 3-butylphthalide,3-butylidene-4-hydrophthalide, 6,7-dihydroxyligustilide and 6,7-epoxyligustilide.

3. The method of claim 1, wherein the phthalide compound has a synergistic effect with 2,3-BPG in the Alzheimer's disease patient.

4. The method of claim 1, which further comprises adjunctly administrating 2,3-BPG to the patient.

5. A method for treating 2,3-BPG metabolic disorder induced morbidities, comprising administering a phthalide compound to a patient having 2,3-BPG metabolic disorder induced morbidities, wherein the phthalide compound substitutes for or cooperates with 2,3-BPG in the patient having 2,3-BPG metabolic disorder induced morbidities to increase the oxygen release efficiency of hemoglobin for the patient.

6. The method of claim 5, wherein the phthalide compound is selected from the group consisting of Z-butylidenephthalide, Z-ligustilide, senkyunolide A, senkyunolide H, senkyunolide I, senkyunolide F, E-butylidenephthalide, E-ligustilide, 3-butylphthalide, 3-butylidene-4-hydrophthalide, 6,7-dihydroxyligustilide and 6,7-epoxyligustilide.

7. The method of claim 5, wherein the phthalide compound has a synergistic effect with 2,3-BPG in the patient having 2,3-BPG metabolic disorder induced morbidities.

8. The method of claim 5, wherein the phthalide compound increases the oxygen release efficiency of hemoglobin in the patient having 2,3-BPG metabolic disorder induced morbidities

9. The method of claim 5, which further comprises adjunctly administrating 2,3-BPG to the patient.
